(19) United States
(12) Patent Application Publication
(10) Pub. No.: US 2017/0304453 A1
(43) Pub. Date: Oct. 26, 2017

(54) THIOL-ENE FUNCTIONALIZED HYDROGELS

(71) Applicants: Massachusetts Institute of Technology, Cambridge, MA (US); InVivo Therapeutics Corporation, Cambridge, MS (US)

(72) Inventors: Timothy O'Shea, Cambridge, MA (US); Alex A. Aimetti, Waltham, MA (US); Robert S. Langer, Newton, MA (US)

(21) Appl. No.: 15/508,166

(22) PCT Filed: Sep. 8, 2015
(86) PCT No.: PCT/US2015/048887

§ 371 Date: Mar. 2, 2017

Related U.S. Application Data
(60) Provisional application No. 62/046,618, filed on Sep. 5, 2014.

(51) Int. Cl. A61K 47/34; A61K 31/721; A61K 9/06; A61K 38/38; A61K 9/00

(52) U.S. Cl. CPC A61K 47/34; A61K 38/38; A61K 9/0024; A61K 9/06; A61K 31/721

(57) ABSTRACT

Disclosed herein are biodegradable hydrogel network containing covalently crosslinked bonds between oligomeric poly(thiol) compounds and oligomeric poly(Michael acceptors). The hydrogels do not substantially change shape upon exposure to aqueous solutions, and as such as suitable for local drug delivery to sensitive tissue areas.

### CROSS-REFERENCE TO RELATED APPLICATION

This application claims priority to provisional application, U.S. Application No. 62/046,618, filed Sep. 5, 2014, the disclosure of which is incorporated herein by reference in its entirety.

### FIELD OF THE INVENTION

The invention is generally directed to biodegradable hydrogel networks which may be used for localized drug delivery.

### BACKGROUND OF THE INVENTION

Synthetic polymer hydrogels, composed of hydrophilic polymers covalently or physically assembled into insoluble infinite networks, are versatile materials with a variety of biomedical uses. While a number of hydrophilic molecules have been used for the fabrication of hydrogels, poly(ethylene glycol) (PEG) hydrogels have been extensively explored in many different in vitro and in vivo applications. Covalently crosslinked PEG hydrogels have been utilized for injectable drug delivery systems, cell carriers for tissue engineering, and bone fillers.

Covalently crosslinked hydrogels have been investigated for local drug delivery systems as they may be loaded with diverse drug types such as small molecules, proteins, carbohydrates and oligonucleotides. Due to the hierarchical structure of the gel, release of hydrophilic drugs is controlled by Fickian diffusion, while hydrophobic drugs are released by gel erosion. Theoretically then, PEG hydrogels permit for spatiotemporal control of the drug release by leveraging controllable material degradation and drug diffusion capacity.

However, while there have been several reports regarding PEG hydrogel technology for drug delivery, several key obstacles must be overcome to obtain a clinically useful PEG-hydrogel network-based drug delivery system. For instance, many PEG hydrogels are prepared using toxic and/or reactive catalysts, which limits their applicability for the delivery of sensitive drugs to sensitive tissue areas. Many PEG hydrogels exhibit either impractical or unpredictable gelation properties, meaning they cannot be reliably produced in a consistent manner. Furthermore, most PEG networks swell substantially upon exposure to physiological media, rendering them unsuitable for placement in enclosed (fixed volume) environments such as those found in neurovascular and cardiovascular systems. Additionally, as the hydrogel is eroded, the release rate of the drug is often increased. As such, most hydrogels provide only limited degree of spatiotemporal control of drug release. Finally, many PEG hydrogels contain non-biodegradable crosslinks, meaning that the material persists for periods well after the last amount of useful drug has been released, which can exacerbate a foreign body response. Each of these limitations must be addressed in order to obtain a PEG hydrogel which can be used clinically for local drug delivery, especially in sensitive tissue areas found in neurological and cardiovascular applications.

It is an object of the invention to provide biocompatible hydrogel materials which can be used to deliver bioactive compounds. It is a further object of the invention to provide biocompatible hydrogel networks which do not substantially swell or otherwise change in size upon exposure to physiological solutions. It is another object of the invention to provide a platform for the synthesis of a variety of biocompatible hydrogel networks with tunable properties.

### SUMMARY OF THE INVENTION

Disclosed herein are covalently crosslinked biocompatible and biodegradable hydrogel networks. The networks contain hydrophilic oligomers which are covalently crosslinked via reversible thiol-Michael addition adducts. The hydrogel networks do not substantially swell or otherwise change in size upon submersion in aqueous media, nor do they swell or change shape as they are degraded under physiological conditions. In certain embodiments, the hydrophilic oligomer is a poly(ethylene glycol).

### BRIEF DESCRIPTION OF THE DRAWINGS

FIG. 1A depicts titration curves for various thiol ethoxylated polyol esters (pH on y-axis, molar equivalents NaOH on x-axis).

FIG. 1B depicts gelation times for various thiol ethoxylated polyol esters with PEGDA 575 (time (seconds) on y-axis, specific hydrogels on x-axis (TMPE-TG, GE-TG, GE-TL, GE-MP and GE-MB, respectively, from left to right).

FIG. 1C depicts rheology curves for various hydrogels (modulus [Pa] on y-axis, time (seconds) on x-axis).

FIG. 2A depicts swelling ratio for various hydrogels over 4-55° C. (Qₘ on y-axis, temperature (Celsius) on x-axis).

FIG. 2B photographically depicts the swelling of TMPE-TL5 over a range of temperatures.

FIG. 2C depicts a comparison of hydrogel equilibrated weight fraction and TEPE lipophilicity at T=25° C. The Qₘ decreases linearly with retention time as measured on a UPLC column (R2=0.9856).

FIG. 2D depicts Qₘ comparisons for PEGDAs of different molecular weights using TMPE-TL and GE-TL TEPEs. Wet mass was weighed following 48 hour incubation at 37° C.

FIGS. 2E and 2F depict percentage hydrogel wet mass changes (compared to the initial cured wet mass) over time for different TEPEs formulated with PEGDA575 (FIG. 2E) and TMPE-TL formulated with different PEGDAs (FIG. 2F).

FIG. 2G depicts FT-IR spectra of TMPE-TL5 hydrogels incubated for designated periods in 1×PBS showing the change in the carboxylate peak (1550-1610 cm⁻¹) resulting from hydrogel ester hydrolysis.

FIG. 3A depicts the cumulative release of different molecular weight FITC-Dextrans (31 kDA, 10 kDa, 20 kDa and 40 kDA) from TMPE-TL5 hydrogels.

FIGS. 3B and 3C depict the cumulative release profiles for the 10 kDa (FIG. 3B) and 40 KDa (FIG. 3C) FITC-Dextran encapsulated within different TEPE/PEGDA 575 hydrogel formulations.

FIGS. 3D and 3E depict the cumulative release profiles for FITC labeled ovalbumin (45 kDa) encapsulated within different TEPE/PEGDA 575 (FIG. 3D) formulations, TMPE-TL and different molecular weight PEGDAs (FIG. 3E).

FIG. 3F depicts a comparison of controlled release of FITC ovalbumin and Alexa Fluor 647 IgG from TMPE-TG5 and TL5 hydrogel formulations.

FIG. 3G depicts the cumulative release profiles for hybrid TMPE-TG/TL5 hydrogels. The first number refers to the percentage of TMPE-TG and second number the percentage of TMPE-TL.

FIG. 3H depicts the influence of the percentage of TMPE-TL on Time to 50% release, T50 (filled symbols) and release constant, k (open symbols). k was linearly related to TMPE-TL percentage while T50 followed an exponential trend.

FIG. 3I depicts the percentage hydrogel wet mass change (compared to the initial cured wet mass) for TMPE hybrid hydrogels. Increasing the ratio of TMPE-TL relative to TMPE-TG resulted in a delay in the onset of terminal hydrogel degradation.

FIG. 4A depicts the results of a QUANTI-Blue colorimetric assay (collated data: n=3 per group, two assay replications). This assay demonstrated that TMPE hydrogels induced minimal SEAP levels and consequently low NF-κB and AP-1 activation.

FIG. 4B depicts the results of a MTS assay comparing the number of live cells for TMPE hydrogels, the cell culture plastic, LPS-EK and alginate controls.

### DETAILED DESCRIPTION OF THE INVENTION

### I. Definitions

As used herein, the term “network” refers to a three dimensional substance having oligomeric strands interconnected to one another by crosslinks.

As used herein, the term “active substance” refers to a compound or mixture of compounds which causes a change in a biological substrate. Exemplary classes of active substances in the medical and biological arts include therapeutic, prophylactic and diagnostic agents. The active substance may be a small molecule drug, a vitamin, a nutrient, a biologic drug, a vaccine, a protein, an antibody or other biological macromolecule. The active substance may also be a fertilizer, a pesticide, an insecticide, an insect repellant, a herbicide or other biological active agent. The active substance may be a mixture of any of the above listed types of compounds.

“Biocompatible” and “biologically compatible”, as used herein, refer to materials that are, along with any metabolites or degradation products thereof, generally non-toxic to the recipient, and do not cause any significant adverse effects to the recipient, at concentrations resulting from the degradation of the administered materials. Generally speaking, biocompatible materials are materials which do not elicit a significant inflammatory or immune response when administered to a patient.

“Biodegradable” and “bioerodible,” as used interchangeably herein, generally refers to a the ability of a material to degrade or erode by enzymatic action or hydrolysis under physiologic conditions to smaller units or chemical species that are capable of being metabolized, eliminated, or excreted by the subject. The degradation time is a function of material composition, morphology, such as porosity, particle dimensions, and environment.

As used herein, the adjective “oligomeric” refers to a compound containing a repeating strand of monomeric subunits.

As used herein, the term “oligomeric poly(thiol)” refers to a compound having a central core bonded to at least two oligomeric units, wherein each oligomeric unit is terminated by a thiol functional group (—SH). The oligomeric poly(thiol) contains at least two thiol functional groups.

As used herein, the term “lipophilic oligomeric poly(thiol)” refers to a compound having a central core bonded to at least three oligomeric units, wherein each oligomeric unit is terminated by a thiol functional group (—SH) or a lipophilic carbon chain. The lipophilic oligomeric poly(thiol) contains at least two thiol functional groups.

As used herein, the term “ethoxylated poly(thiol)” refers to a compound having a central core bonded to at least two poly(ethylene oxide) oligomers, wherein each poly(ethylene oxide) oligomer is terminated by a thiol functional group (—SH). The ethoxylated poly(thiol) contains at least two thiol functional groups.

As used herein, the term “lipophilic ethoxylated poly(thiol)” refers to a compound having a central core bonded to at least three poly(ethylene oxide) oligomers, wherein each poly(ethylene oxide) oligomer is terminated by either a thiol functional group (—SH) or a lipophilic carbon chain. The lipophilic ethoxylated poly(thiol) contains at least two thiol functional groups.

As used herein, the term “oligomeric poly(Michael acceptor)” refers to a compound having a central core bonded to at least two oligomeric unit, wherein each oligomeric unit is terminated by a Michael acceptor. The oligomeric poly(Michael acceptor) contains at least two Michael acceptor groups.

As used herein, the term “lipophilic oligomeric poly(Michael acceptor)” refers to a compound having a central core bonded to at least three oligomeric unit, wherein each oligomeric unit is terminated by either a Michael acceptor or a lipophilic carbon chain. The lipophilic oligomeric poly(Michael acceptor) contains at least two Michael acceptor groups.

As used herein, the term “ethoxylated poly(Michael acceptor)” refers to a compound having a central core bonded to at least two poly(ethylene oxide) oligomers, wherein each poly(ethylene oxide) oligomer is terminated by either a Michael acceptor or a lipophilic carbon chain. The ethoxylated poly(Michael acceptor) contains at least two Michael acceptor groups.

As used herein, the term “lipophilic ethoxylated poly(Michael acceptor)” refers to a compound having a central core bonded to at three two poly(ethylene oxide) oligomers, wherein each poly(ethylene oxide) oligomer is terminated by either a Michael acceptor or a lipophilic carbon chain. The lipophilic ethoxylated poly(Michael acceptor) contains at least two Michael acceptor groups.

As used herein, the term “Michael acceptor” refers to a molecular fragment containing either an alkene or alkyne, wherein at least one of the carbons in the alkene or alkyne is directly bonded to an electron withdrawing group.

As used herein, the term “physiologic fluid” refers to an aqueous solution having a pH approximating that of liquids found in mammalian organisms.

The term “aliphatic group” refers to a straight-chain, branched-chain, or cyclic hydrocarbon groups and includes saturated and unsaturated aliphatic groups, including aromatic rings.

The term “alkyl group” refers straight-chain and branched-chain hydrocarbon groups. Unless specified otherwise, the term alkyl group embraces hydrocarbon groups containing one or more double or triple bonds.

The term “cycloalkyl group” refers hydrocarbon groups which form at least one ring. Unless specified otherwise, the term cycloalkyl group embraces ring systems containing one or more double or triple bonds, and also includes aromatic rings.

As used herein, the term “syneresis” refers to the expulsion of solvent from a hydrogel network with concurrent collapse of the network structure. Syneresis may be evaluated using the swelling ratio (Qₘ), where Qₘ values approaching unity (1), reflect an increasing expulsion of solvent. Syneresis is the transition from a higher to lower Qₘ upon equilibrium under a defined temperature and solvent conditions.

### II. Biodegradable Hydrogel Network

Biocompatible, biodegradable hydrogel networks are described herein. The hydrogel networks are derived from oligomeric strands or chains which have undergone crosslinking. The crosslink bonds include covalent bonds formed from a Michael addition between an oligomeric poly(thiol) and oligomeric poly(Michael acceptor). In order to obtain the biodegradable hydrogel network, it is generally preferred that either the oligomeric poly(thiol) contain at least three thiol functional groups, or the oligomeric poly(Michael acceptor) contain at least three Michael acceptor functional groups. In some embodiments, the oligomeric poly(thiol) contains three thiol functional groups.

In certain embodiments, the biodegradable hydrogel network does not substantially swell or contract when exposed to aqueous solutions, such physiologic fluid. Generally, when the network is submerged in an aqueous solution at physiological pH and temperature (37° C.), the network will undergo syneresis in which no more than 60% of the cured weight is expelled. In certain embodiments, the networks expel no more than 40%, 30%, 20%, 10%, 5%, 2% or even 1% of its cured weight when submerged in such aqueous solutions.

Because the networks are biodegradable, they are eroded when maintained under in vivo conditions. In certain embodiments, the network is substantially degraded within a period of seventy days in vivo, or in other times periods such as 60 days, 50 days, 40 days, 35 days, 30 days, 20 days, 10 days, or even within a period of five days in vivo. In contrast to biodegradable networks disclosed in the prior art, the networks disclosed herein do not substantially swell as they degrade. In certain embodiments, the volume change of the network during degradation in certain formulations does not exceed more than 10% of the initial equilibrated volume and is maintained within this tolerance prior to the commencement of terminal degradation. In other formulations the volume increase due to degradation-associated swelling prior to terminal degradation is less than the initial volume change (i.e. syneresis) observed upon equilibration. Upon commencement of terminal degradation the hydrogel volume decreases until complete dissolution of the network is observed. Network degradation is assessed by measuring the wet and dry masses of the hydrogel network after defined time periods of incubation in physiological buffers.

In certain embodiments, the biodegradable hydrogel networks contain a lipophilic domain, which enables the formation of micelles and or other microstructures within the hydrogel network. The formation of micelles permits the inclusion and delivery of hydrophobic drugs into the network. The network may be depicted by the general structure of Formula (1):

A-Bₙ Formula (1)

wherein A and B are derived from oligomers of Formula (2) and (3). The network is formed by reversible covalent interactions between at least one oligomeric poly(thiol) and at least one oligomeric poly(Michael acceptor). In preferred embodiments, the oligomeric unit in both the poly(thiol) and poly(Michael acceptor) is a poly(ethylene oxide) oligomer.

### Oligomeric Poly(thiol)

The oligomeric poly(thiol) may be a compound of formula (2):

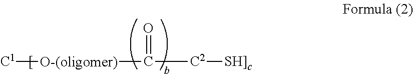

wherein

C¹ represents a C₂₋₁₂ aliphatic moiety;

C² represents a C₁₋₁₂ aliphatic moiety;

(oligomer) represents a hydrophilic oligomeric unit.

Exemplary hydrophilic oligomers include poly(ethylene glycol), carboxymethylcellulose, hyaluronic acid, 2-hydroxyethyl cellulose, poly(vinyl alcohol), dextran, chitin, chitosan, poly(2-hydroxyethyl methacrylate), poly(ethylene glycol)-block-poly(propylene glycol)-block-poly(ethylene glycol), poly(lactide-co-glycolide)-block-poly(ethylene glycol)-block-poly(lactide-co-glycolide), poly(lactide-co-caprolactone)-block-poly(ethylene glycol)-block-poly(lactide-co-caprolactone) and polylactide-block-poly(ethylene glycol)-block-polylactide.

b is 1 or 0; and

c is selected from 2, 3, 4, 5, 6, 7 and 8.

### Lipophilic Oligomeric Poly(thiol)

The lipophilic oligomeric poly(thiol) may be a compound of Formula (2L):

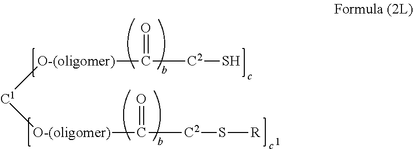

wherein

C¹, C², (oligomer), b and c are as defined for the compound of

Formula (2), and R is a group having the structure:

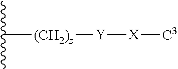

wherein z is 0, 1 or 2;

X is absent, O or NR¹, wherein R¹ is selected from H and C₁₋₆ alkyl;

Y is absent or selected from C═O and SO₂;

C³ is a C₆₋₂₅ lipophilic group;

c¹ is an integer selected from 1, 2, 3, provided that c+c¹≦8.

### Ethoxylated Poly(thiol)

The ethoxylated poly(thiol) may be a compound of Formula (2E):

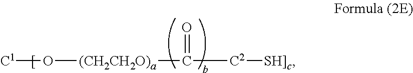

wherein

C¹ represents a C₂₋₁₂ aliphatic moiety;

C² represents a C₁₋₁₂ aliphatic moiety;

a is an integer from 1 to 30;

b is 1 or 0; and

c is selected from 2, 3, 4, 5, 6, 7 and 8.

### Lipophilic Ethoxylated Poly(thiol)

The lipophilic ethoxylated poly(thiol) may be a compound of Formula (2LE):

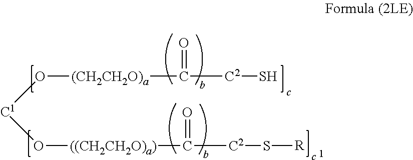

wherein

C¹, C², a, b and c are as defined for the compound of Formula (2E),

and R is a group having the structure:

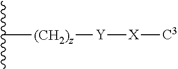

wherein z is 0, 1 or 2;

X is absent, O or NR¹, wherein R¹ is selected from H and C₁₋₆ alkyl;

Y is absent or selected from C═O and SO₂;

C³ is a C₆₋₂₅ lipophilic group;

c¹ is an integer selected from 1, 2, 3 provided that c+c¹≦8.
In certain embodiments of the poly(thiol) compounds, C¹ is selected from one of the following structures:

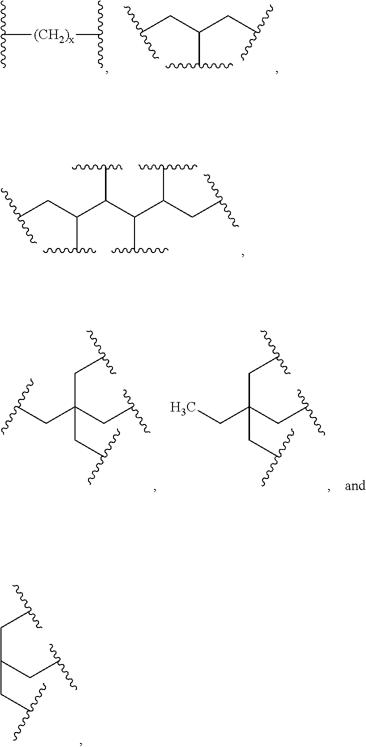

wherein x is an integer from 2 to 6, and the [symbol] symbol depicts a point of connection to the ethoxylated moiety. By way of example, selection of the ethylene aliphatic group (x=2) produces the following embodiment of the compound of Formula (2):

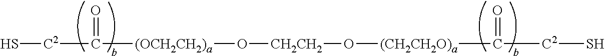

In preferred embodiments of the compound of Formula (2E) or Formula (2LE), a is an integer from 4 to 20, preferably 4 to 12 and most preferably 4-8, b is 1 and c is either 3 or 4. In an especially preferred embodiment, a is 6.

In certain embodiments, C² is:

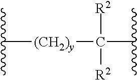

wherein y is from 0 to 12; and
R² is independently selected from hydrogen, C₁₋₆alkyl, including methyl and ethyl, halogen, such as fluorine, chlorine, bromine and iodine, nitro, cyano, trifluoromethyl, or a phenyl ring or aromatic heterocycle such as pyridin-2-yl, pyridin-3-yl, pyridin-4-yl, pyrimidin-2-yl, pyrimidin-4-yl, pyrimidin-5-yl, and morpholin-1-yl, wherein any of the above ring systems may be substituted one or more times with C₁₋₆alkyl, halogen, nitro, cyano and trifluoromethyl.

In other embodiments, C² is a phenyl ring having either the 1,4-, 1,3- or 1,2-substitution pattern:

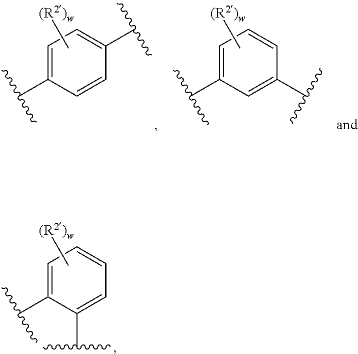

wherein w is selected from 0, 1, 2, 3 or 4 and R^{2′} is independently selected from C₁₋₆alkyl, including methyl and ethyl, halogen, such as fluorine, chlorine, bromine and iodine, nitro, cyano, trifluoromethyl, or a phenyl ring or aromatic heterocycle such as pyridin-2-yl, pyridin-3-yl, pyridin-4-yl, pyrimidin-2-yl, pyrimidin-4-yl, pyrimidin-5-yl, and morpholin-1-yl, wherein any of the above ring systems may be substituted one or more times with C₁₋₆alkyl, halogen, nitro, cyano and trifluoromethyl.

In certain embodiments, the portion of the compound of Formula (2) or (2E) that is [—C(═O)—C²—SH] may be one of the following:

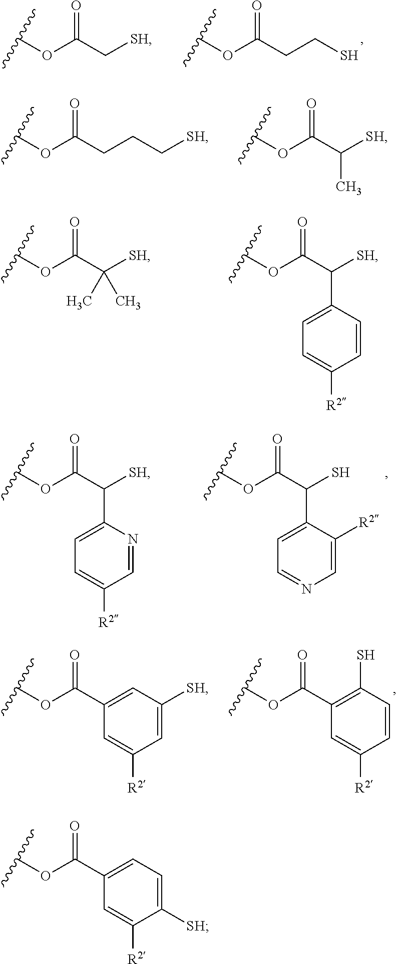

wherein R^{2″} is selected from hydrogen, C₁₋₆alkyl, fluorine, chlorine, bromine, iodine, nitro, cyano and trifluoromethyl. One of ordinary skill will appreciate that the above structural moieties may also be present in the compounds of Formula (2L) and (2LE).

For compounds of Formula (2L) and (2LE), it is preferred that when z is 0, both X and Y are absent, and when z is 2, X is absent, and Y is either C═O or SO₂.

Exemplary C₆₋₂₅ lipophilic groups include linear hydrocarbon chains such those derived from fatty acids, including saturated and unsaturated fatty acids. Exemplary fatty acids include caprylic acid, capric acid, lauric acid, myristic acid, palmitic acid, stearic acid, arachidic acid, behenic acid, lignoceric acid, and cerotic acid. Exemplary unsaturated fatty acids include myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid.

Especially preferred lipophilic groups include the linear, saturated groups represented by the formula:

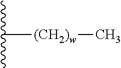

Wherein w is an integer from 5 to 25, preferably 7 to 16, more preferably 7 to 14, and most preferably 9-12.

### Oligomeric Poly(Michael Acceptor)

The oligomeric poly(Michael acceptor) may be a compound of Formula (3A)

C⁴O-(oligomer)-CH₂CH₂—W]ₑ Formula (3)

wherein

C⁴ represents a C₂₋₁₂ aliphatic moiety,

(oligomer) represents a hydrophilic oligomer. Exemplary hydrophilic oligomers include poly(ethylene glycol), carboxymethylcellulose, hyaluronic acid, 2-hydroxyethyl cellulose, poly(vinyl alcohol), dextran, chitin, chitosan, poly(2-hydroxyethyl methacrylate), poly(ethylene glycol)-block-poly(propylene glycol)-block-poly(ethylene glycol), poly(lactide-co-glycolide)-block-poly(ethylene glycol)-block-poly(lactide-co-glycolide), poly(lactide-co-caprolactone)-block-poly(ethylene glycol)-block-poly(lactide-co-caprolactone) and polylactide-block-poly(ethylene glycol)-block-polylactide.

d is an integer from 1 to 14,

e is selected from 2, 3, 4, 5, 6, 7 and 8,

W is a Michael acceptor moiety selected from:

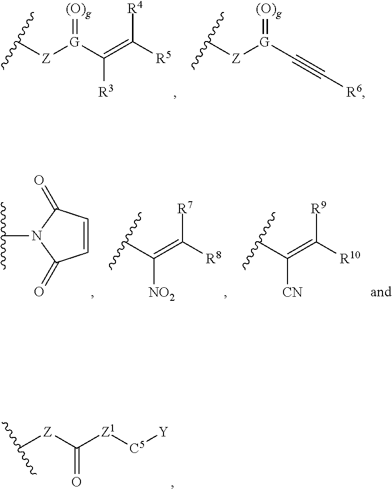

wherein Z and Z¹ independently are either absent or selected from O and NR¹¹;

G is selected from C and S;

wherein when G is C, g is 1, and when G is S, g is either 1 or 2;

R³-R¹¹ is independently selected from H, C₁₋₁₂ alkyl, C₃₋₁₂ cycloalkyl,

wherein two or more of the aforementioned R groups may form a ring;

C⁵ is a C₁₋₁₂ aliphatic group, and

Y is selected from:

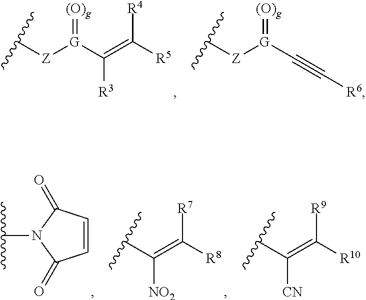

where Z, G, g, and R³-R¹¹ have the aforementioned meanings

### Ethoxylated Poly(Michael Acceptor)

In a preferred embodiment, the oligomeric poly(Michael acceptor) contains a poly(ethylene oxide) unit. The ethoxylated poly(Michael acceptor) may be a compound of Formula (3):

C⁴O—(Ch₂CH₂O)_{d}—CH₂CH₂—W]ₑ Formula (3E)

wherein

C⁴ represents a C₂₋₁₂ aliphatic moiety,

d is an integer from 1 to 14,

e is selected from 2, 3, 4, 5, 6, 7 and 8,

W is a Michael acceptor moiety selected from:

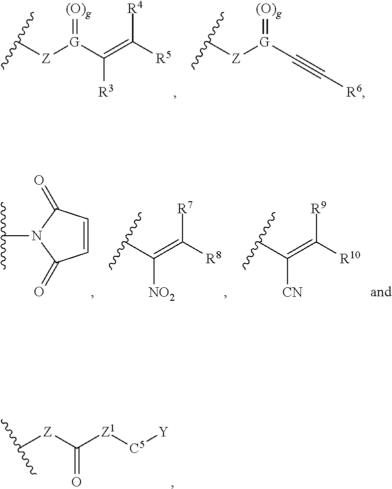

wherein Z and Z¹ independently are either absent or selected from O and NR¹¹;

G is selected from C and S;

wherein when G is C, g is 1, and when G is S, g is either 1 or 2;

R³-R¹¹ is independently selected from H, C₁₋₁₂ alkyl, C₃₋₁₂ cycloalkyl,

wherein two or more of the aforementioned R groups may form a ring;

C⁵ is a C₁₋₁₂ aliphatic group, and

Y is selected from:

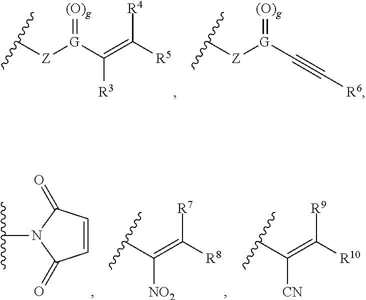

where Z, G, g, and R³-R¹¹ have the aforementioned meanings.

Lipophilic oligomeric poly(Michael acceptors) and lipophilic ethoxylated poly(Michael acceptors), wherein compounds of Formulae (3) and (3E) are derivatized analogously as described for poly(thiol) compounds, are also contemplated as useful oligomeric building blocks for the network. Such compounds are designated herein compounds of Formula (3L) and Formula (3LE)

In certain embodiments, C⁴ is selected from one of the following structures:

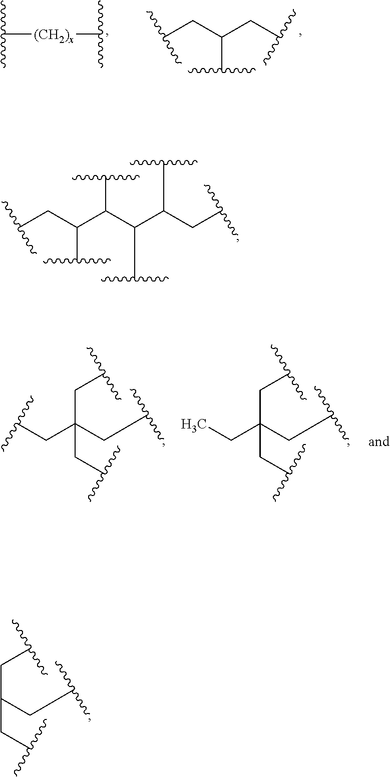

wherein x is an integer from 2 to 6, and the [symbol] symbol depicts a point of connection to the ethoxylated moiety.

In certain embodiments, W has the structure:

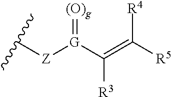

wherein Z, G, and g have the meanings given above, R⁴ and R⁵ are both hydrogen, and R³ is either hydrogen or methyl.

In other embodiments, W is

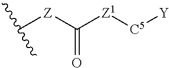

and C⁵ is selected from ethylene or a phenyl ring having a 1,4 substitution pattern.

### Active Substances

The biodegradable hydrogel network may contain one or more active substances, which can be a therapeutic, nutraceutical, prophylactic or diagnostic agent, an herbicide, fertilizer, insecticide, insect repellent, or other material of similar nature. The active substance may be entrapped within the network material or may be directly attached to one or more atoms in the biodegradable hydrogel network through a chemical bond. Representative bond types include covalent and ionic. In a preferred embodiment, the active substance is entrapped within the network.

Generally, the active substance is designed to be released from the biodegradable hydrogel network. Such embodiments are useful in the context of drug delivery. In other embodiments, the active substance is permanently affixed to the network material. Such embodiments are useful in molecular recognition and purification contexts.

In certain embodiments, the active substance is a therapeutic agent. Exemplary classes of agents include, but are not limited to, anti-analgesics, anti-inflammatory drugs, antipyretics, antidepressants, antiepileptics, antiopsychotic agents, neuroprotective agents, anti-proliferatives, such as anti-cancer agents (e.g., taxanes, such as paclitaxel and docetaxel; cisplatin, doxorubicin, methotrexate, etc.), antihistamines, antimigraine drugs, antimicrobials (including antibiotics, antifungals, antivirals, antiparasitics), antimuscarinics, anxioltyics, bacteriostatics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated. These may be vitamins, supplements such as calcium or biotin, or natural ingredients such as plant extracts or phytohormones.

In another embodiment, the therapeutic agent is an immunosuppressive agent. Exemplary immunosuppressive agents include glucocorticoids, cytostatics (such as alkylating agents, antimetabolites, and cytotoxic antibodies), antibodies (such as those directed against T-cell recepotors or I1-2 receptors), drugs acting on immunophilins (such as cyclosporine, tacrolimus, and sirolimus) and other drugs (such as interferons, opioids, TNF binding proteins, mycophenolate, and other small molecules such as fingolimod).

In certain preferred embodiments, the active agent in an antithrombotic such as an anticoagulant or antiplatelet agent. Exemplary antithrombotics include, but are not limited to, coumarins, warfarin, heparin and low molecular weight heparin, factor Xa inhibitors such as rivaroxaban, apixaban and edoxaban, thrombin inhibitors such as hiruin, lepirudin, bivalirudin, agratroba and dabigatran. Exemplary antiplatelet agents include, but are not limited to, abciximab, eptifibatide, tirofiban, oprelvekin, romiplostim and eltrombopag

In a further embodiment, the active agent is used to prevent restenosis in a drug-eluting stent. Exemplary agents include sirolimus (rapamycin), everolimus, zotarolimus, biolimus A9, cyclosporine, tranilast, paclitaxel and docetaxel.

In a further embodiment, the active substance is an antimicrobial agent. Exemplary antimicrobials include antibiotics such as aminoglycosides, cephalosporins, chloramphenicol, clindamycin, erythromycins, fluoroquinolones, macrolides including fidaxomicin and rifamycins such as rifaximin, azolides, metronidazole, penicillins, tetracyclines such a minocycline and tigecycline, trimethoprim-sulfamethoxazole, oxazolidinones such as linezolid, and glycopeptides such as vancomycin. Other antimicrobial agents include antifungals such as antifungal polyenes such as nystatin, amphotericin, candicidin and natamycin, antifungal azoles, allylamine antifungals and echinocandins such as micafungin, caspofungin and anidulafungin.

In other embodiments the bioactive agent is a corticosteroid such as methylprednisolone, methylprednislone sodium succinate and dexamethasone; a chemotherapeutic agent such as paclitaxel, methotrexate, vincristine, doxorubicin, cisplatin; an anesthetic such as lidocaine, bupivacaine, ropivacaine, and chloroprocaine; an analgesic such as morphine, fentanyl, sufentanil, and pethidine; and synthetic growth factor ligands such as LM11A-31.

Generally, a small molecule drug will have a molecular weight less than about 2500 Daltons, preferably less than about 2000 Daltons, even more preferably less than about 1500 Daltons, still more preferably less than about 1000 Daltons, or most preferably less than about 750 Daltons.

In other embodiments, the active substance is a protein or other biological macromolecule. Such substances may be covalently bound to the hydrogel network through ester bonds using available carboxylate containing amino acids, or may be incorporated into hydrogel networks containing olefinic or acetylenic moieties using a thiol-ene radical reaction or conjugate addition. In other embodiments, the biologic is non-covalently associated with the network (e.g., dispersed or encapsulated within). In certain embodiments, the active substance is a growth factor such as fibroblast growth factor (FGF), Brain-derived neurotrophic factor (BDNF), Platelet-derived growth factor (PDGF), Nerve growth factor (NGF), Neurotrophins (NT-3, NT-4), Vascular endothelial growth factor (VEGF), and transforming growth factor b1 (TGF-β1); an enzyme such as chondroitinase, an antibody such an anti-NOGO-A and other proteins such as BA-210 (Cethrin), decorin, and insulin. Carbohydrate based drugs include: sodium hyaluronate and heparan sulfate and nucleotide drugs include: siRNA, mRNA and DNA sequences for temporary or permanent genetic modification.

Additives

The biodegradable network material may also contain other additives, such as plasticizers, stabilizers, preservatives, antioxidants, dyes, pigments, flavoring agents and antistatic agents.

### III. Methods of Making the Network

Methods of Making the Ethoxylated Poly(thiol)

Ethoxylated poly(thiols), including compounds of Formula (2) in which b is equal to one, may generally be prepared by combining an ethoxylated polyol with an excess amount a thiol-containing ester:

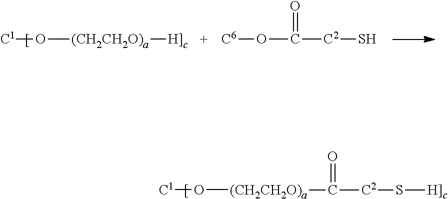

where C¹, C², a and c have the same meanings given above, and C⁶ is hydrogen or an C₁₋₈ aliphatic group. Preferred C⁶ groups include —H, —CH₃, —CH₂CH₃, and vinyl (for the vinyl ester the sulfur atom may be deactivated with a protecting group). The reaction may be carried out either with or without a solvent, preferably without a solvent. Suitable solvents include polar, aprotic solvents such as diethyl ether, tetrahydrofuran, acetone, methylene chloride, DMSO, acetonitrile, DMF, and methylethyl ketone. One of ordinary skill will appreciate the excess amount of thiol ester required to make the ethoxylated poly(thiol) will depend on the number of ethoxy chains present in the ethoxylated polyol.

The reaction may be carried out in the presence of a transesterification catalyst. In certain embodiments, the catalyst is an enzyme, which may be immobilized on a solid support. Exemplary enzymes include lipases such as Pseudomonas fragi 22.39B, Amano Lipase PS, porcine pancreatic lipase, Lipolase 100T, Protease Opticlean M375 (subtilisin from Bacillus licheniformis) Enzymes bound/immobilized on insoluble substrates such as acrylic resin, diatomaceous earth etc. are preferred. Candida antartica Lipase B (“CALB”) immobilized on insoluble acrylic resin such as Novozyme 435 is an especially preferred enzyme for the reaction. In other embodiments, the transesterification catalyst is a small molecule. Exemplary small molecule transesterification catalysts include, but are not limited to, Otera's catalyst, scandium salts and tertiary amines such a dimethylaminopyridine and 1,5,7-triazabicyclo[4.4.0]dec-5-ene.

Under solvent-free conditions, the transesterification may be conducted under mild vacuum. In certain embodiments, the vacuum is less than about 300 mbar, preferably less than about 200 mbar, and even more preferably less than 100 mbar.

In either solvent-free or solvent based transesterification reactions, the reaction may be carried out in the presence of heat. Preferred temperature ranges include 25-100° C., preferably 35-70° C., and even more preferably between 40-60° C.

Compounds of Formula (2L) and (2LE) may be prepared by reacting the compound of Formula (2) or (2E) with a substoichiometric amount of an electrophilic compound having the formula:

Q-(CH₂)_{z}—Y—X—C³

wherein z, Y, X and C³ are as defined above, and Q is a leaving group. Suitable leaving groups include halides such as chloride, bromine, fluorine and iodine, sulfonyl esters such as tosyloxy, mesyloxy and the like. Embodiments in which z is 2 and Y is C═O or SO₂ may be prepared by reacting the compound of Formula (2) or (2E) with a substoichiometric amount of a compound having the formula:

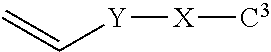

wherein X and C³ are as defined above. The reaction may be carried out in the presence of a polar aprotic solvent such as diethyl ether, tetrahydrofuran, acetone, methylene chloride, DMSO, acetonitrile, DMF, and methylethyl ketone and suitable base such as tertiary amines like triethylamine and diisopropylethyl amine.

The thiol ethoxylated polyol esters of Formula (2E) in which b is equal to zero may generally be prepared by functionalizing the terminal hydroxyl group in an ethoxylated polyol into a leaving group, followed by displacement of the leaving group with an sulfur nucleophile such as potassium thioacetate, and, when necessary, unmasking the thiol group.

The reaction is generally conducted so that substantially all the alcohol groups in the polyol are capped with the thiol-containing group. In certain embodiments, at least 80%, preferably 90% and even more preferably 95% of the alcohol groups are capped. The degree of capping may be determined using conventional techniques such as ¹H NMR spectroscopy.

Ethoxylated poly(thiol) may be purified after the transesterification reaction by conventional techniques, including flash chromatography using silica gel.

Methods of Making the Ethoxylated Poly(Michael Acceptor)

Ethoxylated poly(Michael acceptors) may be obtained from an ethoxylated polyol using conventional chemistries. By way of example, an ethoxylated polyol can be combined with an excess of acryloyl chloride or methacryloyl chloride in the presence of a base in a suitable solvent to yield a multifunctional acrylate or methacrylate ethoxylated polyol. Exemplary bases include trialkylamines such as triethylamine and diisopropylethyl amine, and exemplary solvents include polar, aprotic solvents such as diethyl ether, tetrahydrofuran, acetone, methylene chloride, DMSO, acetonitrile, DMF, and methylethyl ketone. In another embodiment, vinyl (meth)acrylate or ethyl (meth)acrylate can be added in excess to the ethoxylated polyol with CALB under solventless conditions. To synthesize maleimide functionalized ethoxylate polyols in a single reaction step, ethoxylated polyols can be reacted with an excess of p-maleimido phenylisocyanate (PMPI). Finally, to synthesize vinyl sulfone functionalized ethoxylated polyols, the ethoxylated polyols can be reacted with an excess of divinyl sulfone under basic conditions.

Lipophilic poly(Michael acceptors) may be obtained by reacting an a compound of Formula (3) or (3E) under analogous conditions described for the preparation of Lipophilic poly(thiols).

### Biodegradable Hydrogel Network

The biodegradable hydrogel network may be formed by combining at least one oligomeric poly(thiol) with at least one oligomeric poly(Michael acceptor). To form a crosslinked elastic infinite network or hydrogel, either the oligomeric poly(thiol) contains at least three thiol functional groups or the oligomeric poly(Michael acceptor) contains at least three Michael acceptor functional groups. In certain embodiments, the oligomeric poly(thiol) contains three thiol functional groups and the oligomeric poly(Michael acceptor) contains two Michael acceptor functional groups. In other embodiments include the following ratios of oligomeric poly(thiol) functional groups to oligomeric poly(Michael acceptor) groups: 3:3, 4:2. 4:3, and 4:4.

The biodegradable hydrogel is formed by combining the thiol and Michael acceptor components in an aqueous buffer, and incubating the mixture under conditions sufficient to form the hydrogel network. The oligomeric poly(thiol)(s) and oligomeric poly(Michael acceptor)(s) may solubilized individually and then combined, or the components may be combined in bulk and then solubilized in the buffer. In certain embodiments, the oligomers are combined in equal (cured) weight ratios, and in other embodiments an excess of one oligomer (based on cured weight) is added relative to the other.

In certain embodiments, the buffer may be selected from a phosphate monobasic/dibasic solution such as in phosphate buffered saline (PBS), (4-(2-hydroxyethyl)-1-piperazineethanesulfonic acid) (HEPES) solution, tris(hydroxymethyl)aminomethane (Tris) solution or a borate salt solution. In preferred embodiments, the buffer is PBS. Generally, buffer pH values from 7-10 are sufficient to form the hydrogel network. The gelation time of the hydrogel related both to the pka of the thiol groups in the ethoxylated poly(thiol) and the pH of the buffer system. Generally, gelation time depends upon the rate of reaction between the ethoxylated poly(thiol) and ethoxylated poly(Michael acceptor), and thiol groups having lower pKa values exhibit increased gelling rates. Accordingly, the specific gelation time of the gel can be optimized by the selection of more or less acidic thiol functional groups.

The concentration of the buffering agent may be from about 0.5 to about 500 mM, preferably between about 10 to about 50 mM, and more preferably between about 4 to about 5 mM. The buffer solution may further contain salts and sugars to make the solution physiologically isotonic such as, but not limited to, sodium chloride, potassium chloride, dextran, and glucose.

An active ingredient can be incorporated into the network by mixing the active agent into the aqueous buffer solvent at a defined concentration and subsequently solvating both oligomers with the resulting solution/suspension. Alternatively, the active ingredient can be solubilized and mixed into only one of the oligomer solutions. Furthermore, the active ingredient can be added to a mixed hydrogel solution at any time prior to the onset of gelation. Finally, the active ingredient could be included in the incubating media in which the formed hydrogel is allowed to equilibrate in so as to allow the hydrogel to incorporate the active ingredient.

Micelle-containing networks may be prepared using a mixture of oligomers and lipophilic oligomers. Generally, the lipophilic oligomer is present at a weight fraction of total oligomer ranging from 5 to 50%. Preferably the fraction is 15 to 20%. The lipophilic oligomer is combined with the active ingredient in the absence of a solvent. Aqueous buffer is then added to solvate the lipophilic oligomer and active ingredient. This solution is combined with both the oligomeric poly(thiol) and oligomeric poly(Michael acceptor) to initiate gelation. In all cases the number of total number thiol functional groups is equivalent to the number of Michael acceptor groups from the oligomeric poly(Michael acceptor).

After the oligomers are combined, they may be incubated at a temperature of 4-37° C. for a period of 5 minutes to 48 hours to form the hydrogel network. Prior to incubation, the mixture may be poured into a mold so as to obtain a specifically shaped biodegradable hydrogel network. In other embodiments, the biodegradable hydrogel network may be formed into various shapes by machining, cutting, or otherwise sculpting the hydrogel network.

### IV. Methods of Using the Network

The biodegradable networks disclosed herein may be used to deliver bioactive substances to a subject. The network may be fashioned into a depot, implant, mesh, scaffold or other article. The article may be made entirely from the biodegradable hydrogel network, or the article may be made from a combination of the network and one or more other components. The use of additional components permits the construction of articles with greater durability and moldability. Exemplary additional components include bioerodible polymers such as poly hydroxy acids, such as polymers of lactic acid and glycolic acid, polyanhydrides, poly(ortho)esters, polyesters, polyurethanes, poly(butic acid), poly(valeric acid), poly(caprolactone), poly(hydroxybutyrate), poly(lactide-co-glycolide), poly(lactide-co-caprolactone), poly(ethylene-co-maleic anhydride), poly(ethylene maleic anhydride-co-L-dopamine), poly(ethylene maleic anhydride-co-phenylalanine), poly(ethylene maleic anhydride-co-tyrosine), poly(butadiene-co-maleic anhydride), poly(butadiene maleic anhydride-co-L-dopamine) (pBMAD), poly(butadiene maleic anhydride-co-phenylalanine), poly(butadiene maleic anhydride-co-tyrosine), as well as blends comprising these polymers; and copolymers comprising the monomers of these polymers, and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers, blends and mixtures thereof.

The degree of swelling or syneresis is dependent on the difference between the cured weight fraction and the equilibrated weight fraction at a given temperature. For example, for a hydrogel having an equilibrated weight fraction is 47 wt %, any formulation of that hydrogel with a weight fraction less than 47 wt % will synerese to achieve that equilibrated weight fraction. In contrast hydrogels formulated at higher weight fractions will imbibe the solvent to achieve the equilibrated weight fraction. The hydrogel platform can therefore be formulated at designated weight fractions that will achieve the desired amount of syneresis or swelling at a given temperature for the particular application of interest. In one embodiment, the hydrogel may be formulated so that it slightly swells upon exposure to physiologic fluid. Such embodiments may be more easily placed in constrained spaces. As the network swells, it becomes more securely anchored in the space where it was placed.

The release rate of the active substance from the carrier as well as the degradation rate of the carrier itself may be adjusted depending on the particular oligomer units used to prepare the biodegradable hydrogel network Drug delivery in this fashion is especially useful for target sites which are difficult to reach using systemically administered compounds. Exemplary sites include those in the central nervous system, including the brain and spinal cord. Because the biodegradable hydrogel networks do not substantially swell or otherwise change volume as they degrade and release drug, they may be placed in topologically constrained or sensitive areas, such as the spine, brain spinal cord, heart and joints. Specific clinical uses of the biodegradable network include intraparenchymal and intrathecal spinal cord administration for delivery of bioactive substances to treat various aspects of acute, sub-acute and chronic spinal cord injury, intraarticular and intrasynovial delivery of anesthetics, analgesics or anti-inflammatory drugs in orthopedic and spine applications, delivery of chemotherapeutic agents to the brain in cases of brain tumor resection, and delivery of growth factors to areas of ischemic cardiac injury following a myocardial infarction.

### EXAMPLES

Materials: Ethoxylated polyols (glycerol ethoxylate and trimethylopropane ethoxylate), thiol acid ethyl esters (ethyl thiolactate, ethyl thioglycolate, and ethyl 3-mercaptopropionate), γ-Thiobutyrolactone, Lipase B acrylic resin from Candida antarctica (CALB), polyethylene diacrylates (PEGDA) (Mn=575, 700, 1000), activated alumina Brockman I (basic and neutral) were purchased from Sigma-Aldrich (St. Louis, Mo., USA) and TCI America (Portland, Oreg., USA). Phosphate buffered saline, ovalbumin-FITC, dextran-FITC (3 kDa, 10 kDa, 20 kDa, 40 kDa) and IgG-Alex Fluor 647 were purchased from Life Technologies (Grand Island, N.Y.) and Sigma-Aldrich. Filter agent Celite® 545, solvents for flash chromatography (Dichloromethane and Methanol) were purchased from Sigma Aldrich and disposable 80 gram HP Silica Gold Cartridges were purchased from Teledyne Isco. For cell culture work, RAW-Blue™ cell line, QUANTI-Blue™, Normocin, Zeocin, and LPS-EK were purchased from InvivoGen (San Diego, Calif., USA). Dulbecco's Modified Eagle Medium (DMEM, high glucose), heat inactivated fetal bovine serum (HI FBS), and Penicillin-Streptomycin (5,000 U/ml) were purchased from Life Technologies. Endotoxin free water and Corning Costar Ultra-Low attachment 96-well plates were purchased from Sigma-Aldrich.

### Example 1

### Synthesis of Ethoxylated Poly(Thiol)

Ethoxylated polyol (10 mmols˜10 grams) starting material was added to 4A Molecular Sieves (1 gram) into a 100 mL round bottom flask, followed by a five molar excess of thiol acid ethyl ester and Candida antartica Lipase B (CALB) (1 gram). The flask was placed on a magnetic stirrer at 50° C. and purged with Nitrogen gas for 2 hours and then allowed to react overnight under moderate vacuum conditions at 27 inches Hg gauge vacuum or 99 mbar (≈90% vacuum). The reaction was purified by silica flash chromatography on a CombiFlash Rf using a disposable silica column (80 grams) with a dichloromethane/methanol (0-10%) gradient elution method. Purified fractions determined from UV absorbance at 240 nm were combined and dried on a rotary evaporator and under high vacuum before being stored under inert Nitrogen gas at 4° C. Using this technique, the following ethoxylated poly(thiols) were prepared.

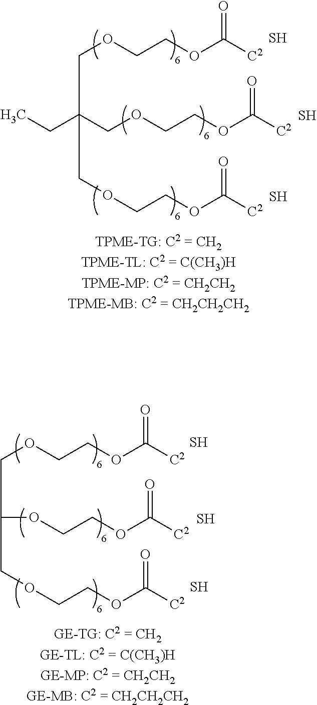

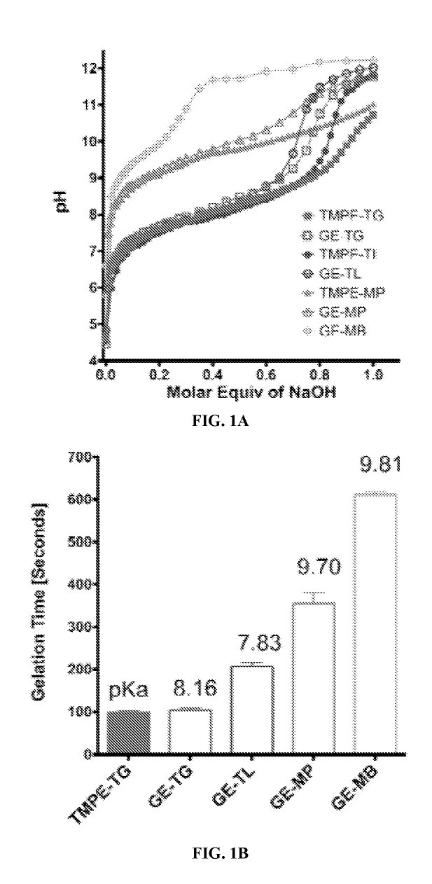

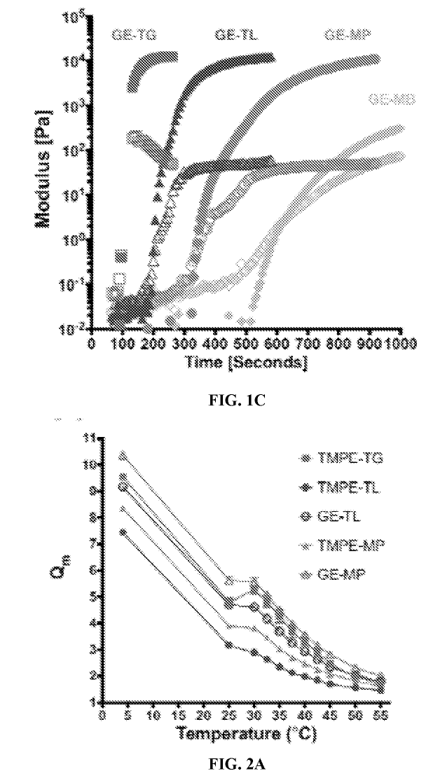

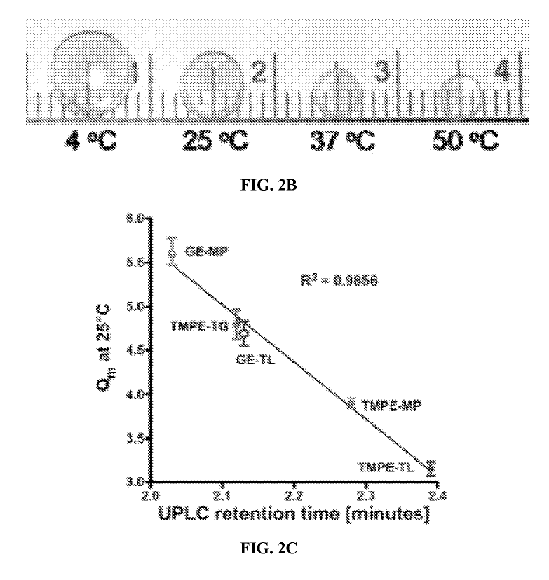

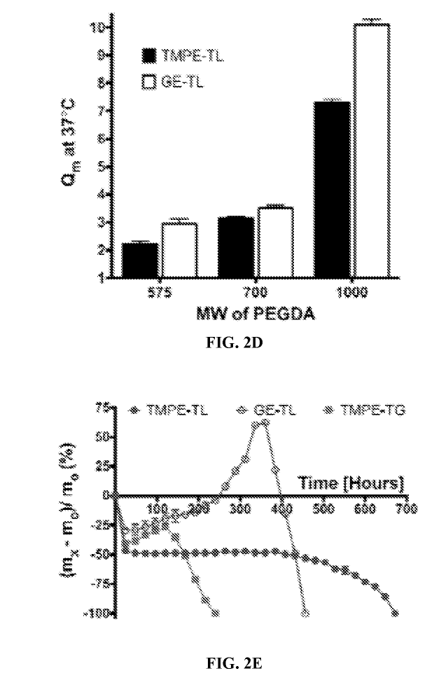

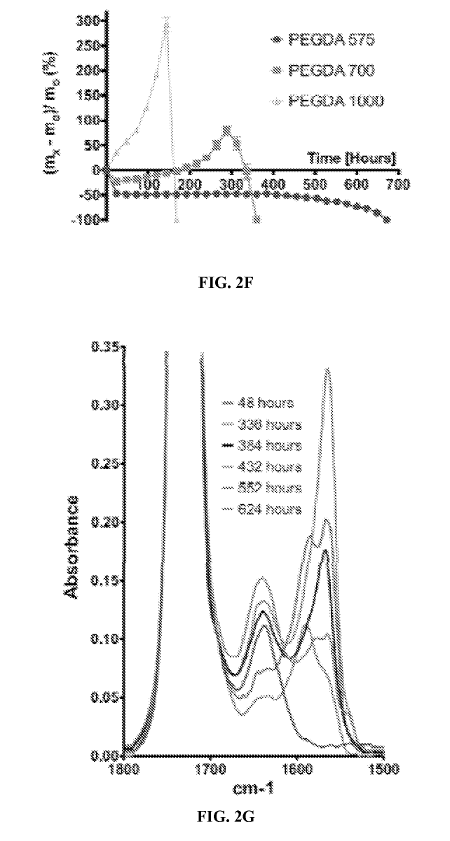

FIG. 1A depicts the relationship between thiol pKa and chemical structure.

### Example 2

### Hydrogel Fabrication

Before formation of the hydrogel network, both oligomers were flashed neat over a small column of activated Neutral Alumina (Brockman I). Individual ethoxylated poly(thiols) from Example 1 and PEG diacrylate (PEGDA) were solubilized with 1×PBS (0.02M, pH=7.4) to form unique solutions of each hydrogel constituent. To enhance the oligomer solubility the solution were placed in a 4° C. environment for 10 minutes. Hydrogels were fabricated upon the addition and mixing of stoichiometric equivalent volumes of the ethoxylated poly(thiols) and PEGDA solutions. In general the volume of PBS added to each hydrogel constituent solution was sufficient to obtain a 25 wt % hydrogel. FIG. 1B depicts the relationship between gelation time and ethoxylated poly(thiol) structure.

A 5943 single column table-top Instron mechanical testing system was used to perform compressive testing on cylindrical hydrogel samples (diameter=3 mm; height=5 mm) that had been allowed to equilibrate in PBS for 48 hours prior to testing. Tests were performed at a rate of 5 mm/min with the force recorded using a 10N load cell and extension of the crosshead used in strain calculations. FIG. 1C depicts differing rheology curves for hydrogels prepared from different TEPE oligomers.

### Example 3

### Hydrogels as a Delivery Device for Active Agents

Dextran Delivery Using Biodegradable Hydrogel Networks

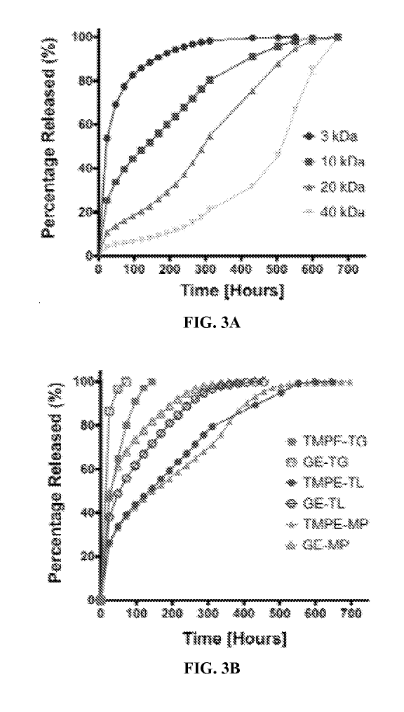

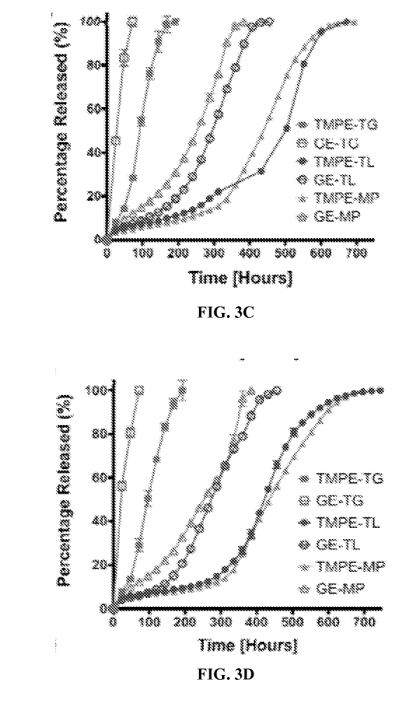

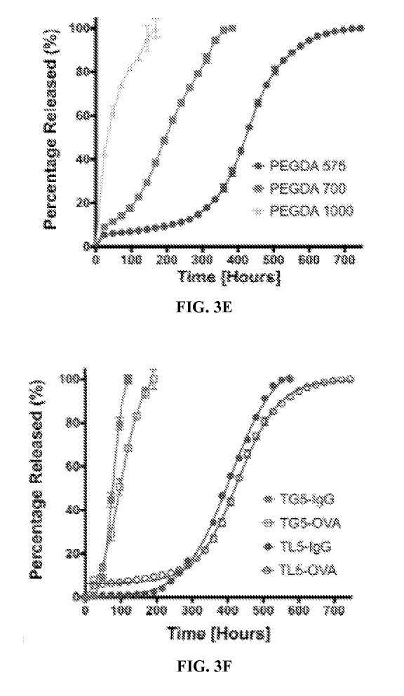

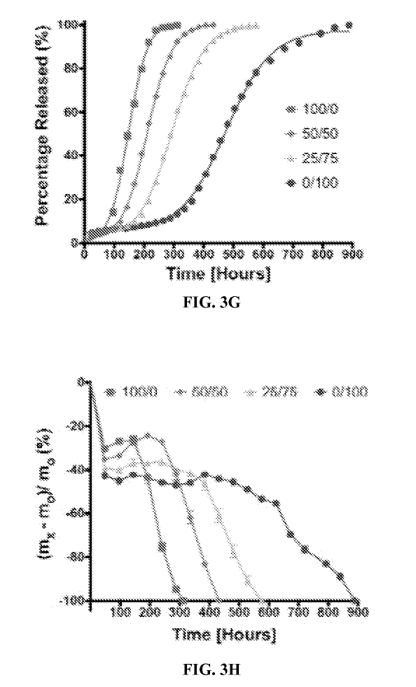

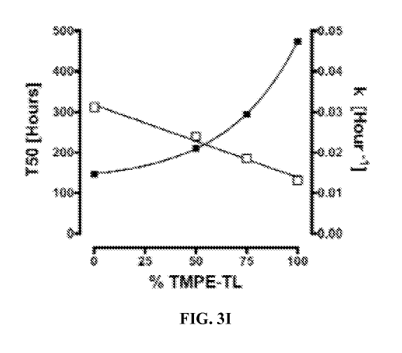

Fluorescein isothiocyanate (FITC) labeled dextrans ranging from 3 kDa to 40 kDa were encapsulated within different the ethoxylated poly(thiol) hydrogels prepared according to Example 1. The dextrans were incorporated by adding the dextran to the hydrogel solution prior to gelation. As depicted in FIGS. 3A-C, the release could be controlled by selection of the particular hydrogel network.

Protein Delivery Using Biodegradable Hydrogel Networks

FITC-labeled ovalbumin (45 kDa) and Alexa Fluor IgG (150 kDa) were encapsulated within different the ethoxylated poly(thiol) hydrogels prepared according to Example 1. The proteins were incorporated by adding the protein to the hydrogel solution prior to gelation. As depicted in FIGS. 3D-E, the release could be controlled by selection of the particular hydrogel network.

### Protein Delivery Using Blends of Biodegradable Hydrogel Networks

Hybrid TMPE-TG and TMPE-TL hydrogel blend formulations were prepared in 50/50 and 25/75 (TMPE-TG:TMPE-TL) ratios. The corresponding oligomeric poly(thiols) were combined in an aqueous buffer, followed by addition of the poly(Michael acceptor). As depicted in FIGS. 3G and 3H, the release profile of ovalbumin could be controlled by modifying the ratio of the primary and secondary thiol containing ethoxylated poly(thiol). By increasing the percentage composition of TMPE-TL (secondary thiol) relative to TMPE-TG, a more prolonged protein release profile and slower hydrogel degradation was observed. Increasing the TMPE-TL (secondary thiol) content also resulted in a delay of the terminal hydrogel degradation, as depicted in FIG. 3I.

### Example 4

### In Vitro Biocompatibility Testing

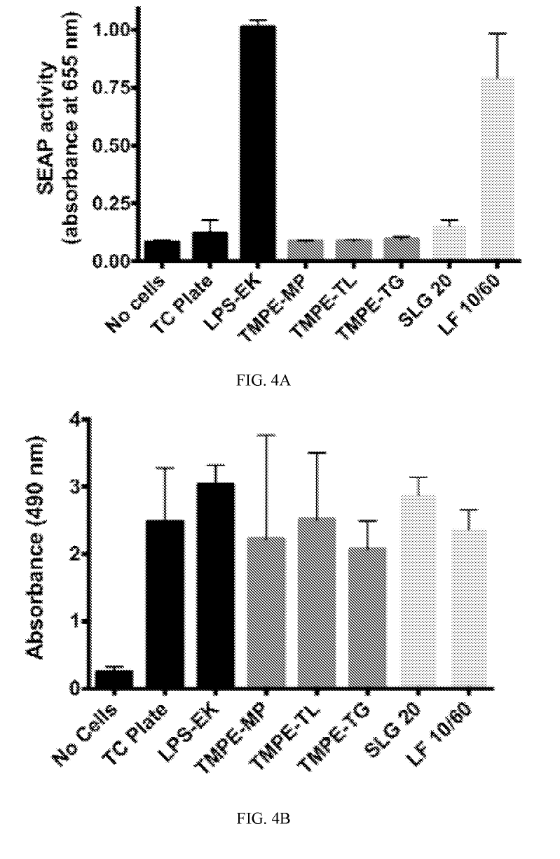

RAW-Blue™ cells were cultured in DMEM medium (4.5 g/L glucose, 2 mM L-glutamine) supplemented with 10% heat inactivated fetal bovine serum, Pen-Strep (50 U/ml), 100 ug/ml Normocin, and 200 ug/ml Zeocin. Lipopolysaccharide from Escherichia coli K12 (LPS-EK Ultrapure 5 μg/ml) was used as a positive control for the PRR activation assay while cell culture treated plastic was used as the negative control. Alginate hydrogels were used as a comparison material and were formulated using pharmaceutical grade Protanal LF10/60 alginate (FMC BioPolymer) and Pronova SLG20 (MW 75,000-220,000 g/mol, >60% G units) (Novamatrix). Alginate hydrogels were formed by adding the alginate solution to a 2.4% barium chloride solution with mannitol with any excess barium chloride being removed through several washes of the hydrogel with HEPES and cell culture media. As depicted in FIGS. 4A and 4B, TEPE-based hydrogels induced minimal SEAP levels and consequently lower NF-κB and AP-1 activation.

1. A biodegradable hydrogel network comprising at least one oligomeric poly(thiol) crosslinked with at least one oligomeric poly(Michael acceptor) having polymer weight fraction of at least 5% but no more than 40%, wherein after submersion in an aqueous solution having a pH of 7.4 at 37° C., the biodegradable hydrogel network is degraded within a period ranging from 5 to 60 days without substantially changing in shape, and wherein the network expels as little as 1% but no more than 60% of its curing weight in the water/buffer.

2. The network of claim 1, wherein the oligomeric poly(thiol) is an ethoxylated poly(thiol) and the oligomeric poly(Michael acceptor) is an ethoxylated poly(Michael acceptor).

3. The network of claim 2, wherein the oligomeric poly(thiol) is an ethoxylated poly(thiol) represented by the compound of Formula (2): 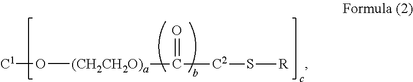
wherein
C¹ represents a C₂₋₁₂ aliphatic moiety;
C² represents a C₁₋₁₂ aliphatic moiety;
R is independently selected from hydrogen or a group having the structure: 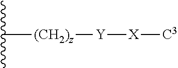
wherein z is 0, 1 or 2;
X is absent, O or NR¹, wherein R¹ is selected from H and C₁₋₆ alkyl;
Y is absent or selected from C═O and SO₂;
C³ is a C₆₋₂₅ lipophilic group;
provided that at least two R groups are hydrogen;
a is an integer from 1 to 30;
b is 1 or 0; and
c is 2, 3, 4, 5, 6, 7 or 8.

4. The network of claim 3, wherein:
C¹ is selected from one of the following structures: 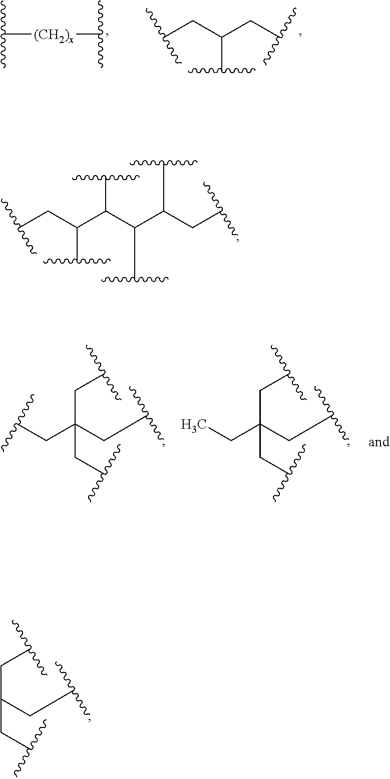
wherein x is an integer from 2 to 6.

5. The network of claim 3, wherein
a is an integer from 4 to 20, preferably 4 to 12 and most preferably 4-8;
b is 1; and
c is either 3 or 4.

6. The network of claim 3, wherein
C² is a divalent phenyl ring, which may be further substituted, or is selected from one of the following structure: 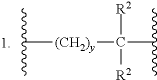
wherein y is from 0 to 12 and R² is selected from hydrogen, methyl and ethyl.

7. The network of claim 3, wherein:
z is 0, and both X and Y are absent.

8. The network of claim 3, wherein:
Z is 2, X is absent, and Y is either C═O or SO₂.

9. The network of claim 3, wherein C³ is a linear hydrocarbon chain.

10. The network of claim 2, wherein the ethoxylated poly(Michael acceptor) represented by the compound of Formula (3):
C⁴O—(CH₂CH₂O)_{d}—CH₂CH₂—W]ₑ Formula (3)
wherein
C⁴ represents a C₂₋₁₂ aliphatic moiety,
d is an integer from 1 to 14,
e is an integer from 2-8,
W is selected from: 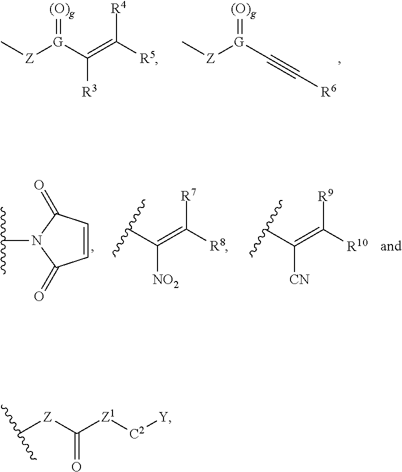
wherein Z and Z¹ independently in each case are either absent or selected from O and NR¹¹;
G is selected from C and S,
wherein when G is C, g is 1, and when G is S, g is either 1 or 2;
R³-R¹¹ are independently selected from H, C₁₋₁₂ alkyl, C₃₋₁₂ cycloalkyl, wherein two or more of the R³-R¹¹ groups may form a ring;
C⁵ is a C₁₋₁₂ aliphatic group, and
Y is selected from: 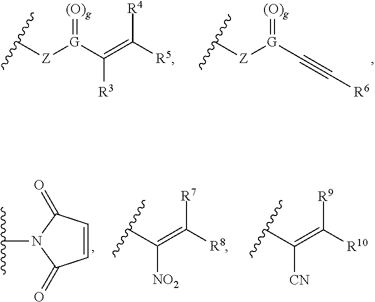
where Z, G, g, and R¹ have the aforementioned meanings,
wherein when e is greater than or equal to 3, one W group may alternatively have the structure: 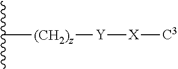
wherein z is 0, 1 or 2;
X is absent, O or NR¹, wherein R¹ is selected from H and C₁₋₆ alkyl;
Y is absent or selected from C═O and SO₂; and
C³ is a C₆₋₂₅ lipophilic group.

11. The network of claim 10, wherein Y has the structure: 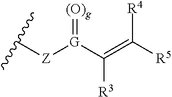
wherein R⁴ and R⁵ are both hydrogen, and R³ is either hydrogen or methyl.

12. The network of claim 10, wherein Y is 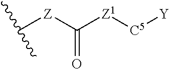
C⁵ is selected from ethylene or a phenyl ring having a 1,4 substitution pattern.

13. The network of claim 1, further comprising at least one bioactive agent.

14. The network of claim 13, wherein the bioactive agent is continuously released over a period of 5 days to 60 days when: (1) the hydrogel is formulated in vitro into a shape defined by a casting mold and then by placing or implanting the casted hydrogel into a sufficient volume of water/buffer/physiological fluid and allowing it to incubate in this volume; (2) the hydrogel is formulated by injecting/depositing/spraying/dripping the mixed precursor solutions into a hydrated environment of water/buffer/physiological fluid and allowing it to cure/form in situ.

15. The network of claim 1, further comprising a lipophilic oligomeric poly(thiol), a lipophilic oligomeric poly(Michael acceptor), or a mixture thereof.
